(19) United States
(12) Patent Application Publication
(10) Pub. No.: US 2022/0322944 A1
(43) Pub. Date: Oct. 13, 2022

(54) OPHTHALMIC INTRAOPERATIVE IMAGING SYSTEM USING OPTICAL COHERENCE TOMOGRAPHY LIGHT PIPE

(71) Applicant: LIV Medical Technology Inc., Baltimore, MD (US)

(72) Inventors: Jin Ung KANG, Glenelg, MD (US); Xuan LIU, Berkeley Heights, NJ (US)

(73) Assignee: LIV Medical Technology Inc., Baltimore, MD (US)

(21) Appl. No.: 17/715,573

(22) Filed: Apr. 7, 2022

Related U.S. Application Data
(60) Provisional application No. 63/173,076, filed on Apr. 9, 2021.

(51) Int. Cl. A61B 5/00; G01B 9/02091; A61B 3/10; A61B 1/07

(52) U.S. Cl. CPC A61B 5/0066; G01B 9/02091; A61B 3/102; A61B 1/07

(57) ABSTRACT

An ophthalmic intraoperative imaging system may include a handheld light probe including a first optical fiber and a second optical fiber. The system may include an illumination light source configured to transmit an illumination beam for intraocular illumination via the first optical fiber of the light probe. The system may include an optical coherence tomography (OCT) light source configured to transmit an OCT beam towards an intraocular region of interest (ROI) via the second optical fiber of the light probe. The system may include an OCT detector configured to detect light reflected by the intraocular ROI. The system may include a processor configured to control the illumination light source and the OCT light source, obtain an OCT signal, obtain a B-mode OCT image of the intraocular ROI by freehand sweeping of the handheld light probe across the intraocular ROI, and control a display to display the B-mode OCT image.

### CROSS REFERENCE TO RELATED APPLICATIONS

This application claims benefit of U.S. Provisional Application No. 63/173,076, filed Apr. 9, 2021, the content of which is incorporated herein by reference in its entirety.

### BACKGROUND

### 1. Field

The present disclosure relates to an ophthalmic intraoperative imaging system, and, more particularly, to an endoscopic optical imaging system using a common-path optical coherence tomography (CP-OCT) light probe for image guided vitreoretinal surgery.

### 2. Description of Related Art

It is estimated that over 180 million people are visually disabled worldwide, and that 40 million to 45 million of these people are blind. While the socio-economic cost of blindness is large, surgical prevention and treatment of vision loss are among the most cost-effective and successful of all medical interventions.

Retinal diseases (e.g., retinal detachment, vitreous hemorrhage, macular puckers, macular holes, and diabetic retinopathy) are the leading causes of blindness, and can be treated effectively through surgery. As the population ages, the need for vitreoretinal surgery continues to grow. However, vitreoretinal surgery remains one of the most challenging surgical procedures because the surgeon has to manipulate a small instrument within a confined and poorly illuminated space that is surrounded by delicate tissues that can be permanently damaged.

During vitreoretinal surgery, a surgeon makes multiple incisions and places trocars at the sclera to insert active microsurgical tools (e.g., 20-25 gauge tools) and a fiber-optic light probe. The intraocular space is illuminated by the light probe. The surgeon uses a stereo operating microscope to visualize surgical instruments and perform surgical procedures. However, the operating microscope provides insufficient depth resolution to identify specific retinal layers, and provides insufficient transverse resolution to reveal microscopic anatomical features.

OCT based on low-coherence interferometry is currently one of the most widely used imaging technologies in ophthalmic diagnosis. However, intraoperative OCT for guiding vitreoretinal surgery requires a time-consuming process to target and orient the scan direction and size, and optimize signal quality. Thus, the use of such OCT imaging inevitably disrupts the surgical procedure.

### SUMMARY

Some example embodiments of the present disclosure provide a handheld light probe (or light pipe) including a multi-mode optical fiber for intraocular illumination, and a single-mode optical fiber for OCT imaging. In this way, some example embodiments herein permit the integration of OCT imaging capability without increasing the dimensions of conventional fiber optic light sources for vitreoretinal surgery.

Similar to a light probe used only for illumination for a microscope, the light probe of the present disclosure may be a manual instrument steered by the surgeon to explore different target areas rapidly within the eye. The light probe guides light for intraocular illumination and can be manually scanned to generate high-resolution and depth-resolved OCT images, and the images can be processed to classified by a real-time machine-learning (ML) algorithm based on a region of interest being identified by the surgeon.

The outcome of vitreoretinal surgery typically depends on the patient's ocular condition, the status of the disease, the surgeon's skill and judgment, and can be improved using instruments that augment the surgeon's capabilities. With OCT-augmented visualization of ocular structures, the light probe of the present disclosure improves treatment effectiveness, minimizes complications, and shortens procedure time for vitreoretinal (or other ophthalmic) surgery. The implementation of OCT functionality with the light pipe does not introduce an additional risk of damaging the retina because the dimension and geometry of the light probe of the present disclosure are substantially similar to light probes that are used only for intraocular illumination during vitreoretinal surgery.

The light probe of the present disclosure permits a surgeon to perform intraoperative OCT imaging whenever the surgeon needs to inspect the depth-resolved structure of the retina without compromising any other ongoing surgical tasks.

The light probe of the present disclosure permits OCT imaging based on a unique “common-path” configuration in which a transmitted OCT beam and light reflected by a region of interest (ROI) share the same probe path. Because of the shared reference and sample arm, CP-OCT allows an OCT signal to be acquired from a thin and flexible light probe with an arbitrary length. CP-OCT is insensitive to the motion of the light probe and is particularly appropriate for manual-scanning OCT imaging. The light probe using the CP-OCT configuration permits the identification of different layers of the retina and pathological areas for assisting vitreoretinal surgery.

The OCT light probe of the present disclosure eliminates the need for a reference arm and a mechanical scanner. Accordingly, the ophthalmic intraoperative imaging system is compact and robust, and the price of the ophthalmic intraoperative imaging system can be significantly lower than conventional OCT systems. The small dimensions and mechanical flexibility of the light probe utilizing CP-OCT permit easy integration of OCT imaging capability with intraocular illumination.

In this way, the example embodiments of the present disclosure provide a light probe that implements OCT imaging and intraocular illumination in a manner that is intuitive, logical, and safe.

According to an aspect of an example embodiment, an ophthalmic intraoperative imaging system includes a handheld light probe comprising a first optical fiber and a second optical fiber, and configured to be inserted into an eye; an illumination light source configured to transmit an illumination beam for intraocular illumination via the first optical fiber of the handheld light probe; an optical coherence tomography (OCT) light source configured to transmit an OCT beam towards an intraocular region of interest (ROI) via the second optical fiber of the handheld light probe; an OCT detector configured to detect light reflected by the intraocular ROI via the second optical fiber of the handheld light probe; and a processor configured to: control the illumination light source to transmit the illumination beam, and control the OCT light source to transmit the OCT beam; obtain an OCT signal based on the light detected by the OCT detector; obtain a B-mode OCT image of the intraocular ROI through freehand sweeping of the handheld light probe across the intraocular ROI; and control a display to display the B-mode OCT image.

According to another aspect of an example embodiment, a method of intraoperatively displaying an optical coherence tomography (OCT) image may include controlling an OCT light source to transmit an OCT beam towards an intraocular region of interest (ROI) via an optical fiber of a handheld light probe that is inserted into an eye of a patient; obtaining an OCT signal based on light reflected by the intraocular ROI and detected by an OCT detector via the optical fiber of the handheld light probe; obtaining a B-mode OCT image of the intraocular ROI by freehand sweeping of the handheld light probe across the intraocular ROI; and controlling a display to intraoperatively display the B-mode OCT image.

According to an aspect of an example embodiment, a handheld light probe for ophthalmic intraoperative imaging may include a first optical fiber configured to optically connect to an illumination light source, and transmit an illumination beam from the illumination light source for intraocular illumination; and a second optical fiber configured to optically connect to an optical coherence tomography (OCT) light source, transmit an OCT beam from the OCT light source towards an intraocular region of interest (ROI), and transmit light reflected by the intraocular ROI towards an OCT detector. The first optical fiber and the second optical fiber each may be a single fiber or a bundle of multiple fibers.

Additional aspects will be set forth in part in the description that follows and, in part, will be apparent from the description, or may be learned by practice of the presented embodiments of the disclosure.

### BRIEF DESCRIPTION OF THE DRAWINGS

The above and other aspects, features, and aspects of embodiments of the disclosure will be more apparent from the following description taken in conjunction with the accompanying drawings, in which:

FIG. 1 is a diagram of an ophthalmic intraoperative imaging system according to an example embodiment;

FIG. 2A is a diagram of a handheld light probe according to an example embodiment;

FIG. 2B is a diagram of a handheld light probe according to another example embodiment;

FIG. 2C is a diagram of a handheld light probe according to another example embodiment;

FIG. 3 is a diagram of a control device according to an example embodiment;

FIG. 4 is a flowchart of an example process for displaying a B-mode OCT image according to an example embodiment;

FIG. 5 is a flowchart of an example process for performing segmentation using a neural network according to an example embodiment; and

FIG. 6 is a diagram of performing segmentation using a neural network according to an example embodiment.

### DETAILED DESCRIPTION

The following detailed description of example embodiments refers to the accompanying drawings. The same reference numbers in different drawings may identify the same or similar elements.

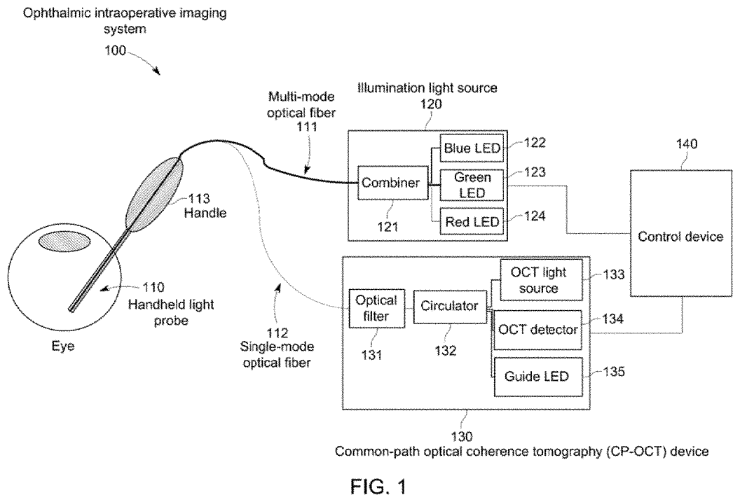

FIG. 1 is a diagram of an ophthalmic intraoperative imaging system according to an example embodiment. As shown in FIG. 1, an ophthalmic intraoperative imaging system 100 includes a handheld light probe 110, an illumination light source 120, a common-path OCT (CP-OCT) device 130, and a control device 140.

The handheld light probe 110 includes a multi-mode optical fiber 111 and a single-mode optical fiber 112. The multi-mode optical fiber 111 and the single-mode optical fiber 112 may be a single fiber or a bundle of multiple fibers. The distal end of the handheld light probe 110 is configured to be inserted into an eye of a patient for intraoperative illumination and imaging. Accordingly, the distal end of the handheld light probe 110 may include a diameter of less than about 1 millimeter. The handheld light probe 110 includes a handle 113 disposed at a proximal end that is configured to be manually manipulated by a surgeon during surgery. For example, the surgeon may perform freehand sweeping of the handheld light probe 110 across an intraocular ROI. According to an example embodiment, an overall or outer diameter of a portion of the handheld light probe 110 that is inserted into the eye may be less than about 1 mm, 950 microns or less, 900 microns or less, 850 microns or less, or 800 microns or less, and an actual optical fiber diameter of the handheld light probe 110 may be between about 200 microns to about 750 microns, about 250 microns to about 750 microns, about 300 microns to about 750 microns, about 200 microns to about 700 microns, about 250 microns to about 700 microns, or about 300 microns and about 700 microns. In this way, the dimensions of the handheld light probe 110 permit the handheld light probe 110 to be inserted into the eye during vitreoretinal surgery for illumination and OCT imaging.

The illumination light source 120 may include a combiner 121, a blue light-emitting diode (LED) 122, a green LED 123, and a red LED 124. The illumination light source is optically connected to the multi-mode optical fiber 111, and is configured to transmit an illumination beam for intraocular illumination via the multi-mode optical fiber 111 of the handheld light probe 110.

The illumination light source 120 may include any number of LEDs to provide white light illumination or any other color illumination. According to an embodiment, the illumination light source 120 may include three or more LEDs having wavelength ranges of 350 nm to 750 nm. The light transmitted by the LEDs may be combined using a multiplexer, and transmitted via the multi-mode optical fiber 111 to the handheld light probe 110. The control device 140 may control the intensity and color of the intraocular illumination provided by the illumination light source 120.

The CP-OCT device 130 may include an optical filter 131, a circulator (and/or coupler) 132, an OCT light source 133, an OCT detector 134, and a guide LED 135. The CP-OCT device 130 is optically connected to the single-mode optical fiber 112 which provides a common transmit and receive optical path such that the CP-OCT device 130 implements CP-OCT.

The OCT light source 133 is configured to transmit an OCT beam through the circulator 132 and optical filter 131 towards an intraocular region of interest (ROI) via the single-mode optical fiber 112 of the handheld light probe 110. The OCT detector 134 is configured to detect light reflected by the intraocular ROI. The optical filter 131 is configured to block light from the illumination light source 120.

The CP-OCT device 130 may use a common-path configuration that does not have a separate reference arm. The OCT light source 133 may be a broadband light source, a swept-source, or the like. According to an example embodiment, the wavelength of the OCT light source 133 may be greater than 700 nm so as to not overlap with the wavelength range of the illumination light source 120. The guide LED 135 transmits light to enable the surgeon to identify the scanning location of the handheld light probe 110, and accurately target the intraocular ROI.

The control device 140 is connected to the illumination light source 120 and the CP-OCT device 130, and is configured to control the illumination light source 120 to transmit the illumination beam, and control the OCT light source 133 to transmit the OCT beam. Further, the control device 140 is configured to obtain an OCT signal based on the light detected by the OCT detector 134, obtain an OCT image of the intraocular ROI based on the OCT signal, and control a display to display the OCT image. The OCT image may be an A-mode image, an M-mode image, a quasi-B-mode image obtained by freehand scanning the light probe, or the like.

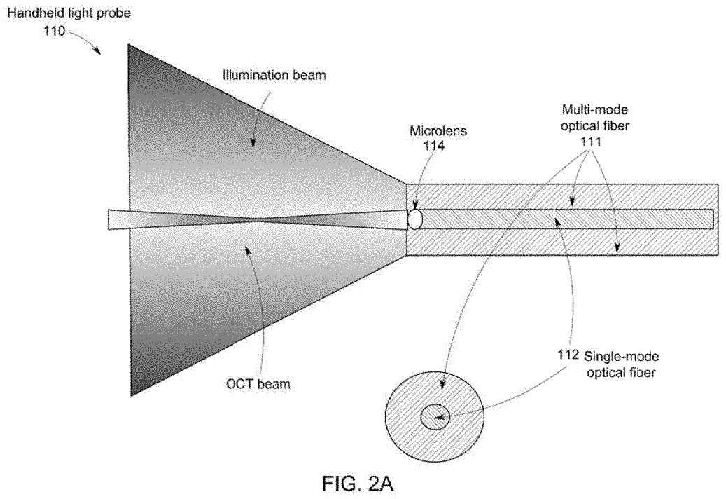

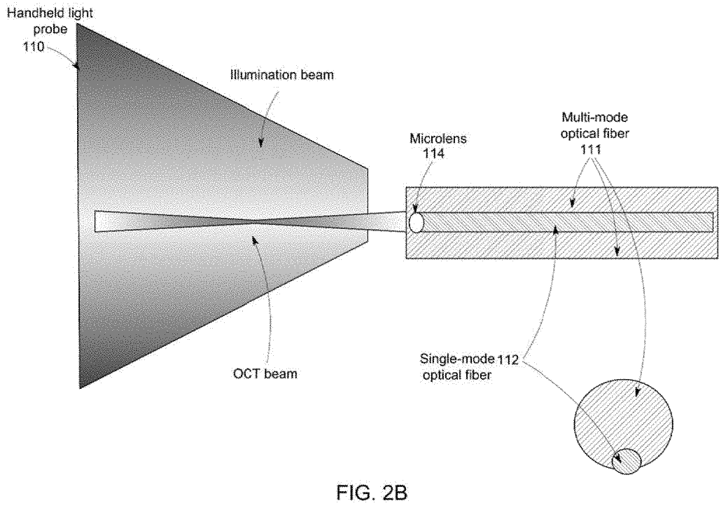

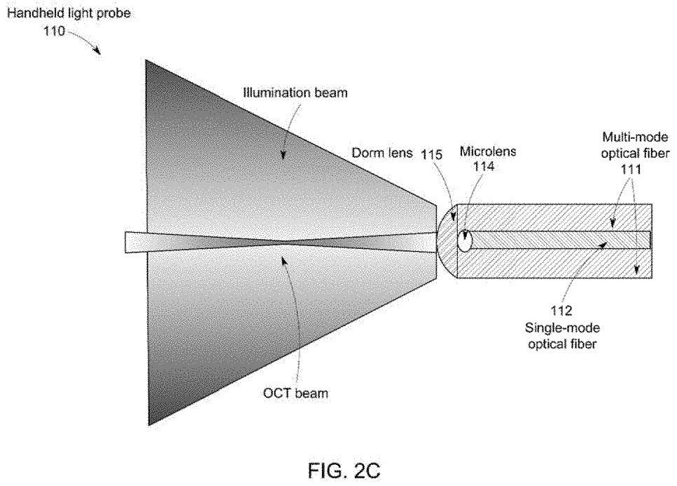

FIG. 2A is a diagram of a light probe according to an example embodiment. As shown in FIG. 2A, the handheld light probe 110 may include the single-mode optical fiber 112 disposed at a center of the handheld light probe 110 in the axial direction of the handheld light probe 110, and may include the multi-mode optical fiber 111 circumferentially disposed around the single-mode optical fiber 112. Further, as shown, the handheld light probe 110 may include a microlens 114 disposed on the single-mode optical fiber 111 near the distal end of the handheld light probe 110 that is configured to collimate or focus the OCT beam towards an intraocular ROI. Light that is reflected by the intraocular ROI is coupled back to the single-mode optical fiber 112 via the microlens 114 and routed back to the OCT detector 134.

According to an example embodiment, the single-mode optical fiber 112 diameter may be less than about 150 microns, about 125 microns or less, about 100 microns or less. The single-mode optical fiber 112 may be fused into the multi-mode optical fiber 111. The multi-mode optical fiber 111 may operate with, or without, any optical elements disposed at the distal end of the handheld light probe 110 for wide-field illumination. The single-mode optical fiber 112 may have either a microlens 114, or no additional optical elements, for imaging. The reference plane may have an epoxy layer or a bare fiber end facet. In this way, the dimensions of the single-mode optical fiber 112 permit the single-mode optical fiber 112 to be integrated with the multi-mode optical fiber 111, which permits the handheld light probe 110 to provide illumination for a microscope via the multi-mode optical fiber 111 and provide CP-OCT imaging via the single-mode optical fiber 112.

FIG. 2B is a diagram of a light probe according to another example embodiment. As shown in FIG. 2B, the handheld light probe 110 may include the single-mode optical fiber 112 that is disposed at a position that is offset from a center of the handheld light probe 110 in the axial direction of the handheld light probe 110. For example, the single-mode optical fiber 112 may be disposed at an edge of the light probe 110 as shown in FIG. 2B.

FIG. 2C is a diagram of a light probe according to another example embodiment. As shown in FIG. 2C, the handheld light probe 110 may include a dorm lens 115 disposed at a distal end of the handheld light probe 110. The spherical dorm lens 115, or similar structure, may obtain a specific illumination angle of the illumination beam and focus the OCT beam.

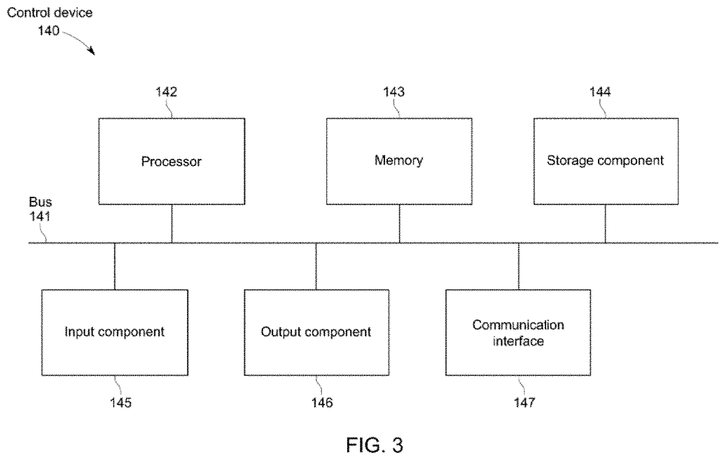

FIG. 3 is a diagram of a control device according to an example embodiment. As shown in FIG. 3, the control device 140 may include a bus 141, a processor 142, a memory 143, a storage component 144, an input component 145, an output component 146, and a communication interface 147.

The bus 141 includes a component that permits communication among the components of the control device 140. The processor 142 may be implemented in hardware, firmware, or a combination of hardware and software. The processor 142 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 142 may include one or more processors capable of being programmed to perform a function.

The memory 143 may include a random access memory (RAM), a read only memory (ROM), and/or another type of dynamic or static storage device (e.g., a flash memory, a magnetic memory, and/or an optical memory) that stores information and/or instructions for use by the processor 142.

The storage component 144 may store information and/or software related to the operation and use of the control device 140. For example, the storage component 144 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 145 may include a component that permits the control device 140 to receive information, such as via user input (e.g., a touch screen display, a keyboard, a keypad, a mouse, a button, a switch, and/or a microphone). Additionally, or alternatively, the input component 145 may include a sensor for sensing information (e.g., a global positioning system (GPS) component, an accelerometer, a gyroscope, and/or an actuator). The output component 146 may include a component that provides output information from the control device 140 (e.g., a display, a speaker, and/or one or more light-emitting diodes (LEDs)).

According to an embodiment, the processor 142 may be configured to control the output component 146 to output a warning (e.g., auditory feedback, a visual notification, etc.) based on the light probe being within a threshold distance of a surface of the retina. In this way, the safety of vitreoretinal surgery may be improved by providing a warning to the surgeon based on the handheld light probe 110 being within the threshold distance to the surface of the retina.

The communication interface 147 may include a transceiver-like component (e.g., a transceiver and/or a separate receiver and transmitter) that enables the control device 140 to communicate with other devices, such as via a wired connection, a wireless connection, or a combination of wired and wireless connections.

The communication interface 147 may permit the control device 140 to receive information from another device and/or provide information to another device. For example, the communication interface 147 may include an Ethernet interface, an optical interface, a coaxial interface, an infrared interface, a radio frequency (RF) interface, a universal serial bus (USB) interface, a wireless fidelity (Wi-Fi) interface, a cellular network interface, or the like.

The control device 140 may perform one or more processes described herein. The control device 140 may perform these processes based on the processor 142 executing software instructions stored by a non-transitory computer-readable medium, such as the memory 143 and/or the storage component 144. A computer-readable medium is defined herein as a non-transitory memory device. A memory device includes memory space within a single physical storage device or memory space spread across multiple physical storage devices.

Software instructions may be read into the memory 143 and/or the storage component 144 from another computer-readable medium or from another device via the communication interface 147. When executed, software instructions stored in the memory 143 and/or the storage component 144 may cause the processor 142 to perform one or more processes described herein.

Additionally, or alternatively, hardwired circuitry may be used in place of or in combination with software instructions to perform one or more processes described herein. Thus, the example embodiments described herein are not limited to any specific combination of hardware circuitry and software.

The number and arrangement of the components shown in FIG. 3 are provided as an example. In practice, the control device 140 may include additional components, fewer components, different components, or differently arranged components than those shown in FIG. 3. Additionally, or alternatively, a set of components (e.g., one or more components) of the control device 140 may perform one or more functions described as being performed by another set of components of the control device 140.

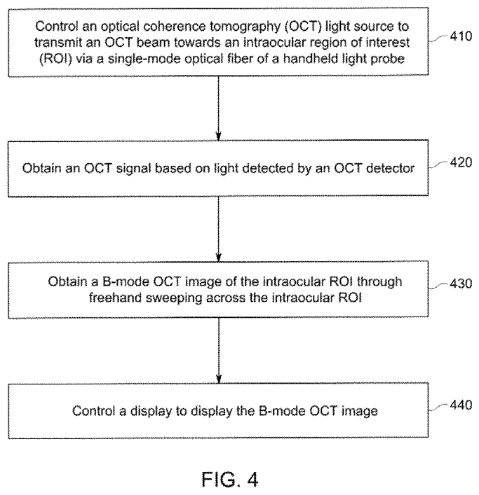

FIG. 4 is a flowchart of an example process for displaying a B-mode OCT image according to an example embodiment. As shown in FIG. 4, the process may include controlling an OCT light source to transmit an OCT beam towards an intraocular region of interest (ROI) via a single-mode optical fiber of a handheld light probe (operation 410). For example, the processor 142 of the control device 140 may control the OCT light source 133 to transmit an OCT beam towards an intraocular ROI via the single-mode optical fiber 112 of the handheld light probe 110.

As further shown in FIG. 4, the process may include obtaining an OCT signal based on light detected by an OCT detector (operation 420). For example, the intraocular ROI may reflect light of the transmitted OCT beam, and the processor 142 may obtain an OCT signal based on the light reflected by the intraocular ROI and detected by the OCT detector 134.

As further shown in FIG. 4, the process may include obtaining a B-mode OCT image of the intraocular ROI through freehand sweeping across the intraocular ROI (operation 430). For example, the processor 142 may obtain an OCT image of the intraocular ROI based on the OCT signal through freehand sweeping, by a user, of the handheld light probe 110 across the intraocular ROI.

According to an embodiment, the processor 142 may obtain a B-mode OCT image that provides discernible features that allow for the effective guidance of vitreoretinal surgery. In this case, the processor 142 may determine the cross-correlation between A-scans obtained with a constant time interval, convert the value of the cross-correlation to lateral displacement, and re-sample the A-scans with a uniform spatial interval to form a distortion-free (or reduced distortion) OCT image. For example, the handheld light probe 110 may be swept across the ROI to generate a quasi B-scan image. The processor 142 may process a detected Fourier domain signal in real-time, and perform fast Fourier transform (FFT) to convert individual spectral interferograms into A-scans. The processor 142 may determine the cross-correlation between the adjacent A-scans to estimate the instantaneous lateral displacement. Further, the processor 142 may re-align the A-scans based on results of the displacement tracking, and obtain distortion-free B-mode images.

As further shown in FIG. 4, the process may include controlling a display to display the B-mode OCT image (operation 440). For example, the processor 142 may control the output component 146 (e.g., a display) to display the B-mode OCT image. In this way, the surgeon may view the OCT image during vitreoretinal surgery.

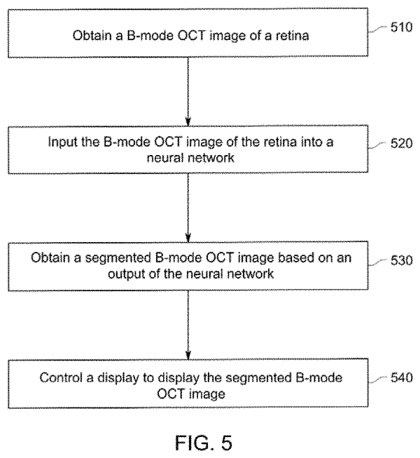

FIG. 5 is a flowchart of an example process for performing segmentation using a neural network according to an example embodiment.

As shown in FIG. 5, the process may include obtaining a B-mode OCT image of a retina (operation 510). For example, the processor 142 may obtain a B-mode OCT image of a retina in a similar manner as described in association with operations 410-430 of FIG. 4.

As further shown in FIG. 5, the process may include inputting the B-mode OCT image of the retina into a neural network (operation 520). For example, the processor 142 may input the B-mode OCT image of the retina into a neural network. The neural network may be a convolutional neural network (CNN), a deep neural network (DNN), a recurrent neural network (RNN), or the like. The neural network may be configured to automatically segment an OCT image such as an A-scan image, an M-scan image, a B-mode image, or the like, of the retina into different layers. The different layers may be a nerve fiber layer, a ganglion cell layer, an inner plexiform layer, an outer plexiform layer, an outer nuclear layer, etc.

The neural network may be configured to identify different retinal layers. Further, the neural network may be configured to differentiate normal and pathological retinal tissue. Further still, the neural network may be configured to quantify a thickness of a retinal layer, and analyze the morphology of the retinal layer for pathological significance. For example, the neural network may be configured to detect an abnormality based on an abnormal thickness or morphology.

As further shown in FIG. 5, the process may include obtaining a segmented B-mode OCT image based on an output of the neural network (operation 530). For example, the processor 142 may obtain a segmented B-mode OCT image based on an output of the neural network.

As further shown in FIG. 5, the process may include controlling a display to display the segmented B-mode OCT image (operation 540). For example, the processor 142 may operate similar to operation 440 in FIG. 4. The processor 142 may control the output component 146 to output information identifying the different layers, information identifying that the tissue is normal, information identifying that the tissue is pathological, or the like. In this way, the surgeon may view the segmented OCT image during vitreoretinal surgery.

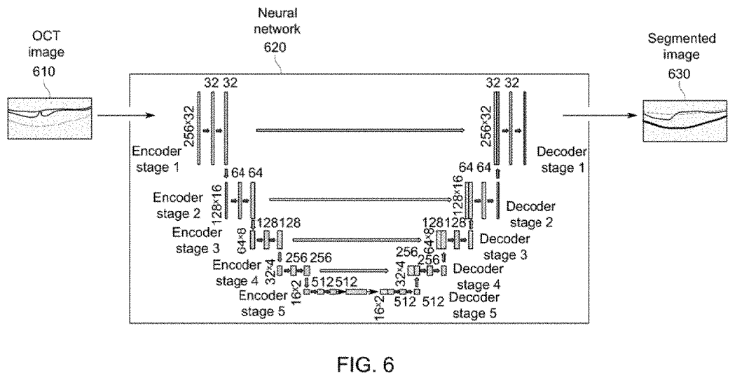

FIG. 6 is a diagram of performing segmentation using a neural network according to an example embodiment. As shown in FIG. 6, and according to an embodiment, a neural network 620 may be a CNN with a u-Net architecture. The processor 142 may input a B-mode OCT image 610 into the neural network 620, and obtain a segmented B-mode image 630 based an output of the neural network 620.

In this way, some example embodiments of the present disclosure provide a handheld light probe including a multi-mode optical fiber for intraocular illumination, and a single-mode optical fiber for OCT imaging. Further, some example embodiments herein permit the integration of OCT imaging capability without increasing the dimensions of conventional fiber optic light sources for vitreoretinal surgery. Accordingly, some example embodiments herein improve vitreoretinal safety, efficacy, and efficiency.

The foregoing disclosure provides illustration and description, but is not intended to be exhaustive or to limit the implementations to the precise form disclosed. Modifications and variations are possible in light of the above disclosure or may be acquired from practice of the implementations.

As used herein, the term “component” is intended to be broadly construed as hardware, firmware, or a combination of hardware and software.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. The term “about” as used herein is intended to include a variation of ±10%, ±9%, ±8%, ±7%, ±6%, ±5%, ±4%, ±3%, ±2%, or ±1% of the recited numerical value. Further, the phrase “based on” is intended to mean “based, at least in part, on” unless explicitly stated otherwise.

What is claimed is:

1. An ophthalmic intraoperative imaging system comprising:
a handheld light probe comprising a first optical fiber and a second optical fiber, and configured to be inserted into an eye;
an illumination light source configured to transmit an illumination beam for intraocular illumination via the first optical fiber of the handheld light probe;
an optical coherence tomography (OCT) light source configured to transmit an OCT beam towards an intraocular region of interest (ROI) via the second optical fiber of the handheld light probe;
an OCT detector configured to detect light reflected by the intraocular ROI via the second optical fiber of the handheld light probe; and
a processor configured to:
control the illumination light source to transmit the illumination beam, and control the OCT light source to transmit the OCT beam;
obtain an OCT signal based on the light detected by the OCT detector;
obtain a B-mode OCT image of the intraocular ROI through freehand sweeping of the handheld light probe across the intraocular ROI; and
control a display to display the B-mode OCT image.

2. The ophthalmic intraoperative imaging system of claim 1, wherein the second optical fiber is disposed in a center of the handheld light probe, and wherein the first optical fiber is circumferentially disposed around the second optical fiber.

3. The ophthalmic intraoperative imaging system of claim 1, wherein the second optical fiber is disposed to be offset from a center of the handheld light probe.

4. The ophthalmic intraoperative imaging system of claim 1, wherein the processor is further configured to:
input the B-mode OCT image into a neural network;
obtain a segmented B-mode OCT image based on an output of the neural network; and
control the display to display the segmented B-mode OCT image.

5. The ophthalmic intraoperative imaging system of claim 4, wherein the first optical fiber is a multi-mode optical fiber, and the second optical fiber is a single-mode optical fiber.

6. The ophthalmic intraoperative imaging system of claim 1, wherein a diameter of the handheld light probe is less than one millimeter.

7. The ophthalmic intraoperative imaging system of claim 1, wherein the handheld light probe further comprises a spherical dorm lens.

8. A method of intraoperatively displaying an optical coherence tomography (OCT) image, the method comprising:
controlling an OCT light source to transmit an OCT beam towards an intraocular region of interest (ROI) via an optical fiber of a handheld light probe that is inserted into an eye of a patient;
obtaining an OCT signal based on light reflected by the intraocular ROI and detected by an OCT detector via the optical fiber of the handheld light probe;
obtaining a B-mode OCT image of the intraocular ROI by freehand sweeping of the handheld light probe across the intraocular ROI; and
controlling a display to intraoperatively display the B-mode OCT image.

9. The method of claim 8, wherein the optical fiber is disposed in a center of the handheld light probe, and wherein another optical fiber for intraocular illumination is circumferentially disposed around the optical fiber.

10. The method of claim 8, wherein the optical fiber is disposed to be offset from a center of the handheld light probe.

11. The method of claim 8, further comprising:
inputting the B-mode OCT image into a neural network;
obtaining a segmented B-mode OCT image based on an output of the neural network; and
controlling the display to display the segmented B-mode OCT image.

12. The method of claim 11, wherein the optical fiber is a single-mode optical fiber.

13. The method of claim 8, wherein a diameter of the handheld light probe is less than one millimeter.

14. The method of claim 8, wherein the handheld light probe further comprises a spherical dorm lens.

15. A handheld light probe for ophthalmic intraoperative imaging, the handheld light probe comprising:
a first optical fiber configured to optically connect to an illumination light source, and transmit an illumination beam from the illumination light source for intraocular illumination; and
a second optical fiber configured to optically connect to an optical coherence tomography (OCT) light source, transmit an OCT beam from the OCT light source towards an intraocular region of interest (ROI), and transmit light reflected by the intraocular ROI towards an OCT detector.

16. The light probe of claim 15, wherein the second optical fiber is disposed in a center of the handheld light probe, and wherein the first optical fiber is circumferentially disposed around the second optical fiber.

17. The light probe of claim 15, wherein the second optical fiber is disposed to be offset from a center of the handheld light probe.

18. The light probe of claim 15, wherein a diameter of the handheld light probe is less than one millimeter.

19. The light probe of claim 15, wherein a diameter of the second optical fiber is less than 150 microns.

20. The light probe of claim 15, further comprising a microlens disposed on the second optical fiber.
